(19) United States
(12) Patent Application Publication
(10) Pub. No.: US 2024/0384321 A1
(43) Pub. Date: Nov. 21, 2024

(54) Microfluidic chip and use thereof for continuous monitoring of bacterium intended for motion, growth and morphology detection

(71) Applicant: National Health Research Institutes, Miaoli County (TW)

(72) Inventors: Chia-Hsien HSU, Miaoli County (TW); Didem RODOPLU SOLOVCHUK, Çayyolu/Ankara (TR)

(21) Appl. No.: 18/667,786

(22) Filed: May 17, 2024

Related U.S. Application Data
(60) Provisional application No. 63/503,353, filed on May 19, 2023.

(51) Int. Cl. C12Q 1/18; B01L 3/00; C12M 1/34; C12M 3/06

(52) U.S. Cl. CPC C12Q 1/18; B01L 3/502715; C12M 23/16; C12M 41/36; B01L 2200/16; B01L 2300/087; B01L 2300/0883

(57) ABSTRACT

The invention relates to a microfluidic chip for motion-based dynamic phenotypic antibiotic drug testing. The microfluidic chip designed with 1000 observation-chambers which physically-traps ˜200 individual bacterium using oil-liquid cutting under certain operational conditions. The oil infusion provides separation of monodispersed pico-chamber volumes within 20 sec. The utility of the microfluidic chip was successfully demonstrated by multi-parametric motion-analysis of ampicillin and gentamicin treated E. coli 25922GFP™ for 21 hours of incubation at 37° C. The large-scanned images are utilized for the enumeration of viable, unculturable, filamentous and dead bacteria isolated in the device. The 2D motion-analysis are found useful for the rapid antibiotic susceptibility testing (AST) within 4 hours. The present invention can also be useful for phenotypic AST of clinical isolates and shed light on the early diagnosis and treatment of antibiotic resistant bacterial diseases in the future.

### CROSS REFERENCE TO RELATED APPLICATIONS

This application claims priority of U.S. Provisional Application No. 63/503,353, filed on 2023 May 19 under 35 U.S.C. § 119(e), the entire contents of which are hereby incorporated by reference.

### BACKGROUND OF THE INVENTION

### 1. Field of the Invention

The present invention relates to a microfluidic chip. Particularly, the present invention relates to a microfluidic chip with two independent units, each unit incorporates 1 main channel, 5 branch channels, 1000 connection channels and 1000 observation chambers and method that monitor motion, growth and morphological changes of isolated bacterium.

### 2. The Prior Art

Antibiotics are medicines used to treat bacterial infections by killing (bactericidal antibiotics) or inhibiting the growth (bacteriostatic antibiotics) of bacteria, to help relieve symptoms of infection and prevent the spread of disease (T. Bollenbach., Current Opinion in Microbiology 27 (2015) 1-9) However, due to overuse or misuse of antibiotics in human and animal medicine as well as agricultural industry, antibiotic resistance can occur, thereby resistance genes can swap to offspring through horizontal gene transfer, and spreads from person-to-person or animal-to-person (S. C. Uzoechi et al., Microscopy and Microanalysis 25(1) (2019) 135-150; P. Dadgostar., Infection and Drug Resistance 12 (2019) 3903-3910). When bacteria become resistant to antibiotics, the treatment of bacterial infections becomes harder and every hour of delay in the treatment may increase the risk of death. It is estimated that more than 700,000 people die each year due to resistant microorganisms. According to the World Health Organization (WHO), antibiotic drug resistant infections are one of the major threats to global health and to overcome this challenge, better understanding of the antibiotic resistance and developing personalized and rapid antibiotic susceptibility tests are of great importance (V. Kandavalli et al., Nature Communications 13(1) (2022) 6215).

The conventional phenotypic antibiotic susceptibility testing (AST) methods, including disk diffusion tests, epsilometer test (E-test) strips, and microdilution methods, which are based on bulky bacterial growth in the absence or presence of antibiotics, require cultivation on the agar plates, and in the liquid culture medium containing tubes or 96 well plates, and necessitates several days for resistance profiling and pathogen identification. As doctors do not have access to fast and point-of-care drug testing methods, they have no choice but to prescribe broad-spectrum antibiotics as a precautionary treatment, resulting in a failure or a delay of a treatment of drug-resistant infections. To reduce the turnaround time for testing and determine an effective personalized drug for the treatment, development of rapid single-bacterium analysis methods is important. Another limitation of conventional phenotypic testing is that it incorporates static information based on growth rate of bulky bacteria regardless of the mechanism of drug resistance. however, a viable bacterium can dynamically adapt to new environments by altering resistance mechanisms. A complementary optical imaging (gram staining, electron microscopy etc.) method can be useful to collect information of the morphology (shape, size, and membrane structure), abundance and localization of certain proteins due to antibiotic pressure. However, it lacks the capability to provide dynamic data without integrating microfluidics.

Microfluidic devices provide a controllable environment for bacterial growth in order to detect time-dependent changes in the morphology, motility, viability and growth (D. Rodoplu et al., Talanta 230 (2021) 122291; D. Rodoplu et al., Microchemical Journal 178 (2022) 107390; O. Scheler et al., Scientific Reports 10(1) (2020) 3282). There are several studies for antibiotic drug testing of individual bacterium. For instance, Postek and Garstecki developed a high-throughput microfluidic droplet platform and utilized fluorescence-based detection for antibiotic drug testing (W. Postek et al., Accounts of Chemical Research 55(5) (2022) 605-615). However, this method has several limitations including lack of providing single-bacterium imaging and leading false positive results due to penetration of viability dye to the neighboring droplets that does not include bacteria. Roslon et al. developed a mass-sensitive graphene drum platform to detect the nano motion of flagella of individual bacterium that enables antibiotic testing in 40 min (I. E. Rosloń et al., Nature Nanotechnology 17(6) (2022) 637-642). However, this technique is lack of providing growth rate, displacement or speed of bacteria.

To date, time lapse microscopy has been utilized to reveal the changes in morphology, motility, and gene expression of bacterium. However, capturing direct changes of individual bacterium has been challenging due to rapid motion of bacterium in a bulky medium. With the integration of single-bacterium microfluidics, bacterium motion tracking can be a revolutionary tool to study dynamic phenotypic heterogeneity of bacteria as different bacterial cells within a population can exhibit different levels of susceptibility to antibiotics.

Take together, current devices possess challenges for isolating and tracking viable, highly motile pathogens. Thus, the present invention provides a microfluidic chip for individual-bacterium isolation and enrichment platform that can be useful for phenotypic characterization and antibiotic susceptibility testing.

### SUMMARY OF INVENTION

In one aspect, the present invention relates to a fluidic chip for microorganism detection
and analysis comprising:
a top layer,
wherein the top layer comprises:
at least one main channel facilitates the flow of a sample solution,
a plurality of branch channels is configured for facilitates the flow of oil,
a plurality of observation chambers is utilized for observing a single sample in the sample solution,
a plurality of connection channels connects the branch channel and the observation chambers.

In some embodiments, the main channel further comprising:
a) an inlet through which the sample solution is introduced to the fluidic chip.
b) an outlet through which the sample solution is removed from the fluidic chip.

In some embodiments, the main channel splits into 2-8 branch channels.

In some embodiments, the main channel spans width ranging from 1-2 mm.

In some embodiments, the branch channel span width ranging from 5-150 μm and height ranging from 5-20 μm.

In some embodiments, the connection channel span width of 0.25-50 μm.

In some embodiments, the observation chamber is configured for trapping the single microorganism.

In some embodiments, the observation chamber can be one of the following shapes: rectangular prism-shaped, pentagonal prism-shaped, heptagonal prism-shaped, octagonal prism-shaped, triangular prism-shaped, square prism-shaped, cylindrical prism-shaped and trapezoidal prism-shaped.

In some embodiments, the observation chambers spans height ranging from 1-14 μm.

In some embodiments, the observation chamber spans 50-100 μm in width and 50-200 μm in length for a rectangular prism shape chamber.

In some embodiments, the observation chamber spans ranging from 100-300 μm diameter for a cylindrical shaped chamber.

In some embodiments, the fluidic chip spans 6-20 mm in length.

In some embodiments, the top layer the top layer is composed of the following materials: polydimethylsiloxane (PDMS), polycarbonate (PC), polymethyl methacrylate (PMMA), polyolefin copolymer (POC), polystyrene (PS), polypropylene (PP) plastics, and hydrogel.

In some embodiments, the bottom layer is disposed on the glass bottom layer.

In some embodiments, the sample solution is composed of any one of the following species or combinations thereof: bacteria, fungi, archaea, protists, eukaryotes.

In another aspect, the present invention relates to a method for utilizing a fluidic chip comprising:
a) Filling fluidic chip with a growth medium and the sample solutions at 0.0001-0.1 ml/h and 0.0001-0.3 ml/h flow rate, respectively,
b) Conducting oil-liquid cutting by oil infusion by a syringe pump at 0.001-0.1 ml/h flow rate within 15-60 sec to trap 0 or 1 microorganism inside the observation chamber.

In some embodiments, the method further comprises monitoring the sample solution at temperature-controlled chamber with 37° C.

In some embodiments, the method further comprises, prior to operation, the top layer is immersed in 1-10XPBS overnight and 500 μl 1-10XPBS is added to the bottom layer.

In some embodiment, the sample solution comprises bacteria with a concentration ranging from 5×10³-5×10⁴ CFU/ml to trap 0 or 1 microorganism inside the observation chamber.

### BRIEF DESCRIPTION OF THE DRAWINGS

Non-limiting embodiments of the present invention will be described by way of example with reference to the accompanying drawings, which are schematic and are not intended to be drawn to scale. In the drawings, each identical or nearly identical component illustrated is typically represented by a single numeral. For the purposes of clarity, not every component is labeled in every drawing, nor is every component of each embodiment of the invention shown where illustration is not necessary to allow those of ordinary skill in the art to understand the invention. In the drawings:

FIG. 1 is the illustration of the microfluidic chip design. Theres is main channel (20), branch channel (21), and observation chamber (23).

FIG. 2 is the illustration of the assemble microfluidic chip. The microfluidic chip (10) includes a top layer (11) having an inlet (31) and an outlet (32), and a bottom layer (12).

FIG. 3 is the illustration of the channels on microfluidic chip. Theres is main channel (20), branch channel (21) and observation chamber (23).

FIG. 4 is the top view of the channels. Theres is branch channel (21), connection channel (22), and observation chamber (23).

FIG. 5 is the top view of the channels loading with culture medium (41).

FIG. 6 is the top view of the channels loading with sample solution (42).

FIG. 7 is the top view of the channels conducting oil-liquid cutting (43).

FIG. 8 is the cross-sectional view of the microfluidic chip. Theres is branch channel (21), connection channel (22), and observation chamber (23).

FIG. 9 is the cross-sectional view of the microfluidic chip loading with culture medium (41).

FIG. 10 is the cross-sectional view of the microfluidic chip loading with sample solution (42).

FIG. 11 is the cross-sectional view of microfluidic chip conducting oil-liquid cutting (43).

FIG. 12 shows the effect of microfluidic chip length on the capture efficiency of single viable bacterium.

FIG. 13 shows the effect of trapped bacterium number on gentamicin susceptibility.

FIG. 14 shows time-dependent X-Y-t Micrograph of bacteria motion. A) individual bacterium motion. B) monoclonal heterogeneity after 4 hours in microfluidic chip.

FIG. 15A-B show antibiotic susceptibility testing of Escherichia coli (E. coli) GFP. A) under ampicillin treatment. B) under gentamicin treatment.

FIG. 16A-D is the Schematic drawing of the miniature clone picking setup which could be operated in a small biosafety cabinet. A) process flow diagrams showing an example method for clone picking according to an aspect of the present disclosure. B) Perspective-view of the setup system. C) Determining the single clone in observe channel. D) Picking up a single clone with a robot arm.

FIG. 17A-17F is the potential microfluidic chip designs.

### DETAILED DESCRIPTION

All the features disclosed in this specification may be combined in any combination. An alternative feature serving the same, equivalent, or similar purpose may replace each feature disclosed in this specification. Thus, unless expressly stated otherwise, each feature disclosed is only an example of a generic series of equivalent or similar features.

### Microfluidic Chip

The present disclosure relates to microfluidic chip and methods that monitor bacterium motion behavior. The transparent microfluidic chip that can limit the Z-shift of object position in order to facilitate imaging long trajectories of bacterium with high magnification, thereby assisting segmentation for 2D-motion analysis using both fluorescence and phase-contrast microscopy.

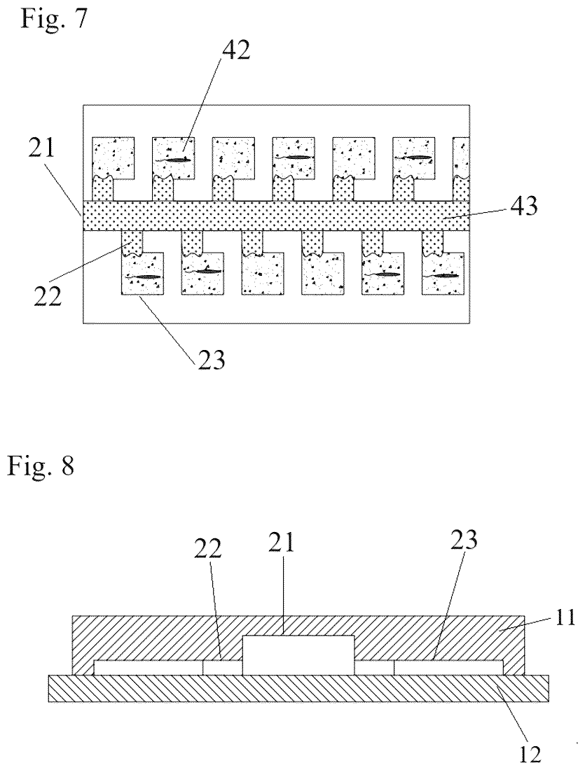

The microfluidic chip consisting of a polydimethylsiloxane (PDMS) top layer and a glass coverslip bottom layer (a 35 mm cell culture dish). The microfluidic chip comprising 5 branch channels incorporating 200 connection channels and 200 observation chambers in each main channel, which physically-traps individual bacterium in 3.7 μm depth of observation chamber using synthetic oil-liquid cutting. The Z-depth of the branch channel is designed for easy oil-water cutting, while shallow observation chambers with a thickness of 3.7 μm and dimensions of 50×100×3.7 μm (W×L×H) are designed to trap bacteria in closed chamber, facilitating optical imaging (see FIG. 7)

### Sample Handling and Analyzing

Escherichia coli (E. coli) (ATCC 25922 GFP™) was chosen as a model bacterium. Ampicillin and gentamicin were chosen as model antibiotic drugs. The operational parameters were optimized to isolate 0 or 1 bacterium in each chamber. For the determination of minimum inhibitory concentration (MIC), fluorescence intensity of the sample was measured via inverted fluorescence microscope at 2 hours intervals, during 6 hours of incubation in a temperature-controlled chamber at 37° C. The time-dependent morpho-structural variabilities of bacterium were characterized by a software. The MIC value obtained for bulky bacteria was found comparable with the E-test results. 0.25 μg/ml GM inhibits bulky E. coli GFP growth on agar plates and microfluidic chip. On the contrary, 0.1 μg/ml GM inhibits growth of ˜90% E. coli GFP in the microfluidic chip, and resistant subpopulation continue growing after 4 h. The present invention shed light on breakthrough development in bacterial resistance research and rapid drug testing applications.

### Imaging and Data Analysis

Micrographs and real-time movies were recorded under Nikon Eclipse Ti-2 inverted fluorescence microscope using 40× objective and Q-Imaging camera (RETIGA-400 Fast 1394DC, Tucson, USA). For phenotypic characterization of bacterium, microfluidic chip was placed in a temperature-controlled chamber of an inverted fluorescence microscope at 37° C., and real-time movies of bacterium with 60 sec durations were recorded during 5 hours of incubation at 1-hour intervals. Images were analyzed with NIKON Analysis and MATLAB software.

### Example 1: Microfluidic Chip Design, Fabrication and Assembly

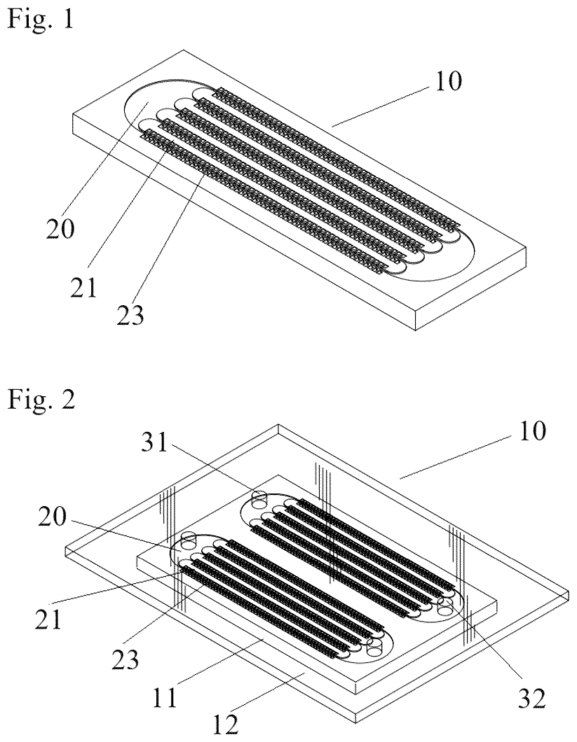

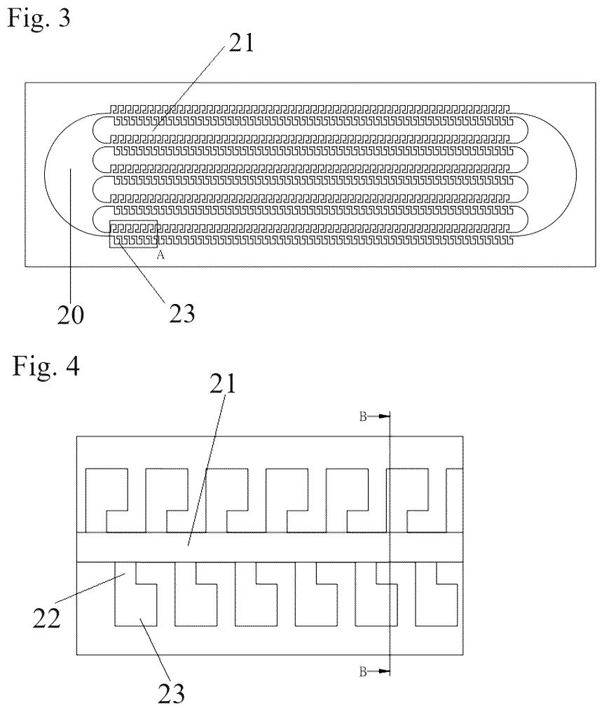

In this and the following examples, the microfluidic chip consists of a top layer (10) and a bottom layer (12). The top layer is made with polydimethylsiloxane (PDMS), designed using AutoCAD software and constructed on the bottom layer (12). The bottom layer is a glass-bottom petri dish (α plus, bottom glass dish, Taiwan), which has an inner glass with diameter and thickness of 21 and 0.17 mm, respectively (FIG. 2). The top layer (10) comprises 5 branch channels (21) incorporating 200 connection channels (22) and 200 observation chambers (23) in main channel (20) (FIG. 1). The main channel (20) is disposed on the top layer. The branch channel (21) is connected to the observation chambers (23) via a connection channel (22).

The preliminary studies showed that physical dimensions of the branch channel with a width of 50 μm and a height of 14 μm, is suitable for easy oil flow. However, 14 μm height of the chamber limits measuring the motility of bacterium due to difficulties of focusing and tracking of individual bacterium.

The branch channel (21) is configured with a width ranging from 5-150 μm and a height ranging from 5-20 μm, and the height/width ratio to 0.34. The observation chambers (23) are dimensioned at 50×100×3.7 μm (W×L×H), with a height/width ratio of 0.03 to confine bacteria within the enclosed channel, facilitating optical imaging (refer to FIG. 7). Observation chamber can be rectangular prism-shaped, pentagonal prism-shaped, heptagonal prism-shaped, octagonal prism-shaped, triangular prism-shaped, square prism-shaped, cylindrical prism-shaped and trapezoidal prism-shaped.

Connection channel (22) is devised to permit single microorganisms to traverse into the observe channel (21). The width of the connection channel ranges from 0.25-50 μm according to the size of microorganisms provided in Table 1-3.

The microchannel and microstructure are manufactured by a cast molding process, a hot-pressing process, a laser etching process, a soft lithography process or the like. For the preparation of SU-8 masters, 5 g negative epoxy resist (SU-8-50, GELEST INC., USA) was dropped on a silicon wafer, then spin-coated at 3000 rpm for 35 s. The SU-8 coated wafers were baked at 65° C. and 95° C. for 5 and 15 min. For photolithographic patterning, a photomask is aligned to the wafer, then exposed to ultraviolet (UV) light (365 nm) at a dose of 290 mJ/cm². For the post-baking process, wafers were placed on hotplates at 65° C. and 95° C. for 1 and 4 min. After the post-baking step, wafers were placed in the developer solution for 6 min, and dried using N₂ gas. The surface of the SU-8 wafers was modified with silane overnight.

The SYLGARD® 184 silicone elastomer base and curing agent were mixed at a volume ratio of 10:1 for 1 min. Then, 20 g of this solution was poured onto silane-coated masters, degassed under vacuum, and baked in an oven at 65° C. overnight. The PDMS layer was peeled off from the SU-8 surface carefully and trimmed according to the microfluidic contour. The inlet-outlet openings were punched using a 1 mm diameter of punch tool. For the microfluidic chip assembly, the PDMS layer and glass surfaces were exposed to oxygen plasma at 100W for 12 s, bring into contact and incubated at 65° C. to achieve irreversible bonding between the surfaces.

### Example 2: Microfluidic Chip Operation

In this and the following examples, prior to use, the microfluidic chip were sterilized inside a laminar flow hood exposing UV light for 30 min. Following this, microfluidic chip was cleaned by dripping 1 μl 75% ethanol in the inlet (31), and channel was degassed with the flow of sterilized 1XPBS using a syringe pump at 1 ml/h flow rate.

E. coli 25922GFP™ and Muller-Hinton growth medium are chosen as model bacterium and culture medium.

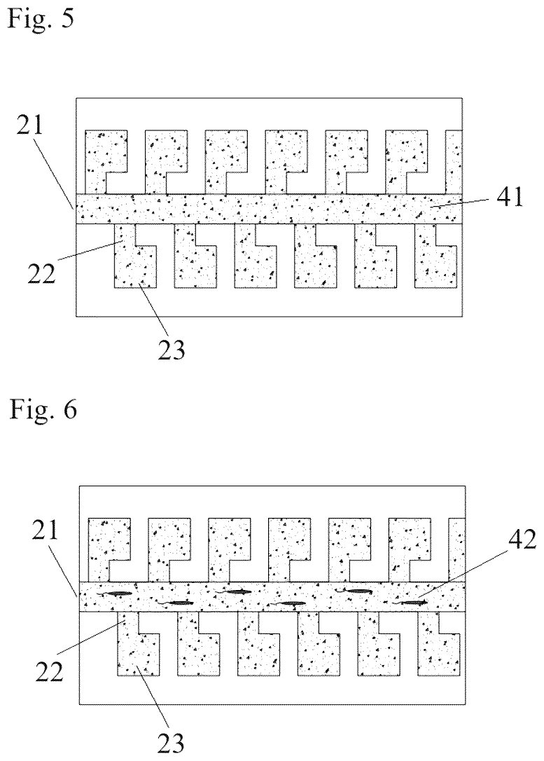

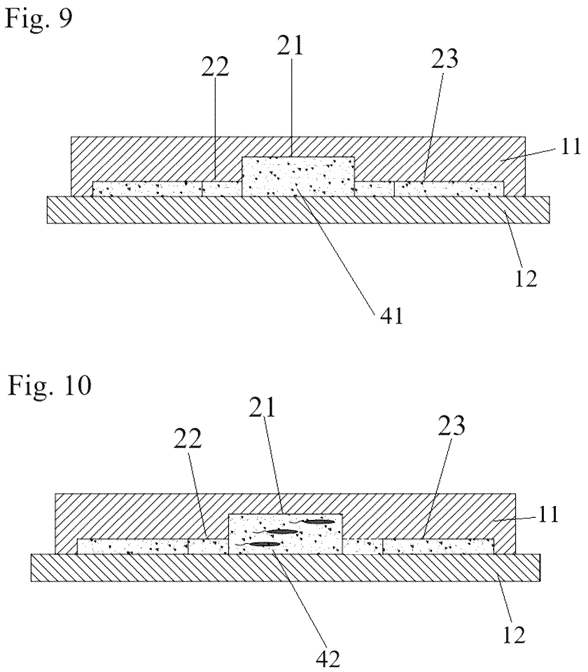

Step 1, microfluidic chip filled with growth medium (41) and bacteria solution (42) at 0.1 ml/h and 0.03 ml/h flow rate, respectively (FIG. 5-6; FIG. 9-10). In this and the following examples, loading 5×10⁴ CFU/ml bacteria with 0.03 ml/h flow rate for 1 min was found suitable for trapping ˜200 individual bacterium in observe channel (21) of the microfluidic chip (FIG. 10)

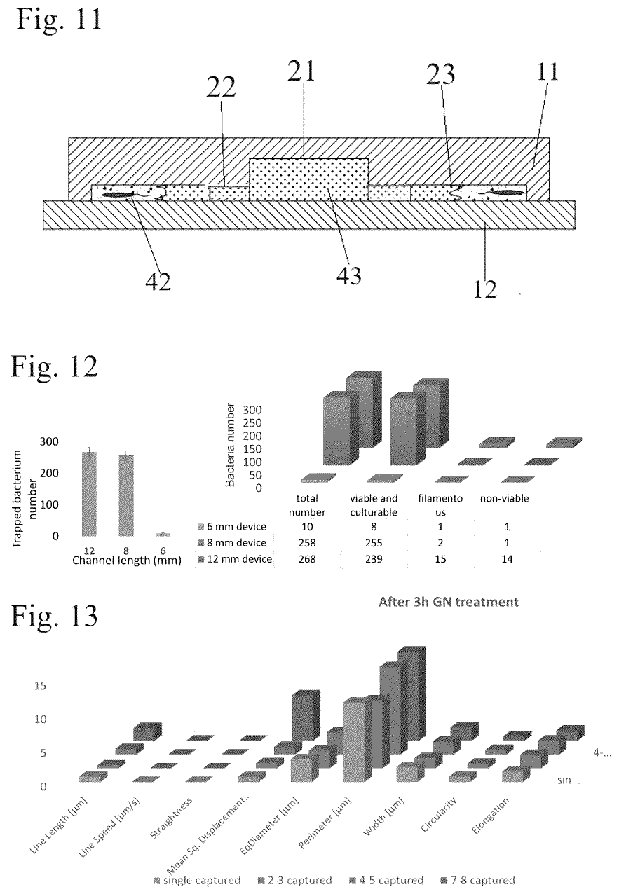

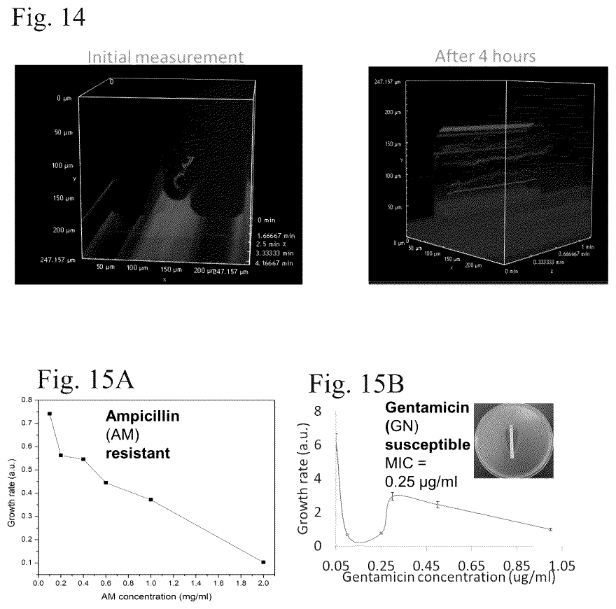

Step 2, oil-liquid cutting (43) was conducted by synthetic oil (10W-40) infusion by a syringe pump at 0.01 ml/h flow rate to obtain volumetrically identical chamber isolation within 20 sec (FIG. 7; FIG. 11). The 16.8 μm height of branch channels (21) and 0.01 ml/h oil infusion for 20 sec provide homogenous oil-liquid cutting, and humidity channel maintains 13 picoliter volume for long-incubation periods as well. The microfluidic chip isolates volumetrically identical 13 picoliter volumes in total 2000 observation chambers. In order to prevent evaporation of picoliter liquid medium during long-term incubation, the top layer (11) is immersed in 1XPBS overnight prior to operation, and 500 μl 1XPBS is added to the bottom layer (12) after assembling microfluidic chip.

Synthetic oil is a non-polar hydrocarbon that London forces and dipole-dipole interactions are interacting between molecules. It has a lower surface tension and a higher viscosity than Mueller Hinton growth medium, which is dissolved in water. Owing to the momentum transfer to the capillary microfluidic chip walls, an increase in the hydraulic resistance limits the liquid flow.

Step 3, The morpho-structural changes and Dynamic phenotypic analysis of bacterium were evaluated by microscopy methods. The large-scanned device images are utilized for the enumeration of viable, unculturable, filamentous and dead bacteria isolated in the device. Dynamic analysis also allows conducting viability testing, toxicity testing and AST of microorganism. In this and the following examples, E. coli is monitored during the first 6 hours and after 21 hours of incubation at 37° C. inside a temperature-controlled chamber placed on the microscope-stage.

In some embodiment, the present microfluidic chip can be operated with manual pipetting when the height of the observation chambers and channels is above 12 μm.

In some embodiment, the present microfluidic chip can be also operated with a vacuum pump connected to the outlet of the microfluidic chip while solution is introduced with a reservoir on the inlet.

### Example 3: Optimization of the Microfluidic Chip and Method

Under intensive physical and chemical stress factors, bacteria cells enter an injured or dormant state, resulting in a non-motile and non-dividing phase of live bacteria. In order to prevent operational stressors on microorganism, the design of microfluidic chip and operational parameters are optimized to create stress-free culture microenvironment (FIG. 12). The morpho-structural changes of bacterium were evaluated by fluorescence imaging. The large-scanned device images are utilized for the enumeration of viable, unculturable, filamentous and dead bacteria isolated in the device. As a model bacterium, E. coli expressing green fluorescence genes were monitored during 24 hours of incubation using a time-lapse fluorescence microscope. As shown in FIG. 12, 8 mm length of microfluidic chip allows capturing 98% viable and culturable bacteria. When the length of the channel increases, the flow resistance and physical stress on bacteria increase, resulting with an increase on filamentous bacteria number (FIG. 12).

TABLE 1
Morpho-structural parameters of some pathogenic bacteria and recommended height of observe channel (HOC)

| | | | | | | | | | Approximate |
|---|---|---|---|---|---|---|---|---|---|
| | | | | | | | | | height of |
| | | | | | | | | Approximate | observation |
| | Length | Width | | | Flagella | Flagella | Speed | Average | chamber of |
| | Range | Range | | | Length | Speed | Range | Speed | SII-chip |
| Bacterium | (μm) | (μm) | Circularity | Motility | (μm) | (μm/s) | (μm/s) | (μm/s) | (HOC) |
| Escherichia coli | 1.5-2.0 | 0.25-1.2 | 0.8-0.9 | Peritrichous | 10-20 | 20-30 | 10-80 | 30-40 | 3-4 |
| and uropathogenic | | | | flagella, | | | | | |
| E. coli (UPEC) | | | | occasionally | | | | | |
| | | | | twitching | | | | | |
| | | | | motility | | | | | |
| Bacillus | 3-5 | 0.25-1.0 | 0.7-0.9 | Subpolar | 8-15 | 10-20 | 2-50 | 20-30 | 5-6 |
| subtilis | | | | flagella, | | | | | |
| | | | | occasionally | | | | | |
| | | | | gliding | | | | | |
| | | | | motility | | | | | |
| Pseudomonas | 1-5 | 0.5-1 | 0.7-0.9 | Polar | 10-20 | 10-20 | 1-100 | 10-20 | 3-6 |
| aeruginosa | | | | flagellum, | | | | | |
| | | | | twitching | | | | | |
| | | | | motility | | | | | |
| Salmonella | 2-5 | 0.7-1.5 | 0.7-0.9 | Peritrichous | 10-20 | 50-80 | 20-100 | 60-70 | 4-7 |
| | | | | flagella, | | | | | |
| | | | | occasionally | | | | | |
| | | | | darting | | | | | |
| | | | | motility | | | | | |
| Proteus | 1-3 | 0.5-1 | 0.7-0.9 | Peritrichous | 10-15 | 20-30 | 5-40 | 20-25 | 4-5 |
| mirabilis | | | | flagella, | | | | | |
| | | | | swarming | | | | | |
| | | | | motility | | | | | |
| Klebsiella | 1-2 | 0.5-1 | 0.7-0.9 | Peritrichous | 10-15 | 20 | 10-80 | 20-30 | 3-4 |
| pneumoniae | | | | flagella, | | | | | |
| | | | | occasionally | | | | | |
| | | | | twitching | | | | | |
| | | | | motility | | | | | |
| Mycobacterium | 2-4 | 0.2-0.5 | 0.5-0.9 | Non-motile | N/A | N/A | N/A | N/A | 3-4 |
| tuberculosis | | | | | | | | | |
| Methicillin- | 0.5-1.5 | 0.5-1.5 | 0.8-0.9 | Non-motile | N/A | N/A | N/A | N/A | 3-4 |
| resistant | | | | | | | | | |
| Staphylococcus | | | | | | | | | |
| aureus (MRSA) | | | | | | | | | |

TABLE 2
Morpho-structural parameters of fungi and recommended height of observe channel

| | | | | | | | | Approximate | | |
|---|---|---|---|---|---|---|---|---|---|---|
| | Length | Width | | | Flagella | Flagella | Speed | Average | | |
| | Range | Range | | | Length | Speed | Range | Speed | | |
| Fungi | (μm) | (μm) | Circularity | Motility | (μm) | (μm/s) | (μm/s) | (μm/s) | Disease(s) | HOC |
| Saccharomyces | 3-5 | 1-1.5 | 0.9-1 | Uniflagellate | 3-5 | 20-25 | N/A | N/A | N/A | 5-7 |
| cerevisiae | | | | | | | | | | |
| Candida | 5-10 | 2-4 | 0.9-1 | Uniflagellate | 10-15 | 20-25 | N/A | N/A | Candidiasis | 8-12 |
| albicans | | | | | | | | | | |
| Aspergillus | 2-4 | 2-3 | 0.9-1 | Non-motile | N/A | N/A | N/A | N/A | Aspergillosis | 4-6 |
| fumigatus | | | | | | | | | | |
| Penicillium | 2-4 | 1-2 | 0.8-0.9 | Non-motile | N/A | N/A | N/A | N/A | N/A | 4-5 |
| chrysogenum | | | | | | | | | | |
| Neurospora | 7-10 | 2-3 | 0.9-1 | Biflagellate | 5-10 | 20-25 | N/A | N/A | N/A | 10-11 |
| crassa | | | | | | | | | | |
| Cryptococcus | 4-6 | 1-2 | 0.9-1 | Uniflagellate | 3-5 | 5-10 | N/A | N/A | Cryptococcosis | 5-7 |
| neoformans | | | | | | | | | | |
| Trichophyton | 4-6 | 2-4 | 0.8-0.9 | Non-motile | N/A | N/A | N/A | N/A | Dermatophytosis | 5-7 |
| rubrum | | | | | | | | | | |
| Candida | 3-5 | 1-2 | 0.9-1 | Uniflagellate | 5-10 | 20-25 | N/A | N/A | Candidiasis | 5-7 |
| glabrata | | | | | | | | | | |
| Rhizopus oryzae | 10-20 | 5-10 | 0.8-0.9 | Non-motile | N/A | N/A | N/A | N/A | Mucormycosis | 20-21 |
| Histoplasma | 2-5 | 2-3 | 0.8-0.9 | Non-motile | N/A | N/A | N/A | N/A | Histoplasmosis | 5-7 |
| capsulatum | | | | | | | | | | |

TABLE 3
Morpho-structural parameters of Archaea Species and recommended height of
observe channel

| | Length | Width | | | |
|---|---|---|---|---|---|
| Archaea Species | Range (μm) | Range (μm) | Circularity | Motility | HOC |
| Methanocaldococcus | 0.6-1.5 | 0.2-0.5 | Rod-shaped | Non-motile | 1.2-2 |
| jannaschii | | | | | |
| Halobacterium | 1-10 | 0.2-2 | Irregular shape | Motile | 3-12 |
| salinarum | | | | (using | |
| | | | | multiple | |
| | | | | polar | |
| | | | | flagella) | |
| Methanosarcina mazei | 1-5 | 0.5-1 | Irregular shape | Non-motile | 3-6 |
| Pyrolobus fumarii | 1.5-3.5 | 0.2-0.5 | Irregular shape | Non-motile | 3-4 |
| Thermoproteus tenax | 1-5 | 0.5-1 | Rod-shaped | Non-motile | 3-6 |
| Sulfolobus | 0.5-2 | 0.2-1 | Irregular shape | Non-motile | 1.2-3 |
| acidocaldarius | | | | | |
| Nanoarchaeum equitans | 0.4-0.5 | 0.2-0.3 | Irregular shape | Non-motile | 0.5-2 |

TABLE 4
Doubling time of bacteria and fungi
to aid phenotypic identification

| | Doubling |
|---|---|
| Bacteria | Time (min) |
| Escherichia coli | 20 |
| Uropathogenic E. coli (UPEC) | 22 |
| Bacillus subtilis | 30 |
| Pseudomonas aeruginosa | 60-90 |
| Salmonella | 20-30 |
| Proteus mirabilis | 25-50 |
| Klebsiella pneumoniae | 30-40 |
| Mycobacterium tuberculosis | 600-800 |
| Methicillin-resistant | 30-40 |
| Staphylococcus aureus (MRSA) | |
| | Doubling |
| Fungi | Time (hrs) |
| Aspergillus fumigatus | 6-8 |
| Candida albicans | 1-3 |
| Cryptococcus neoformans | 6-10 |

Under optimum stress-free conditions, the microfluidic chip was found useful for distinguishing morpho-structural variances of viable bacterium after an antibiotic treatment. The optically transparent and ultra-low depth microfluidic chip enables high-resolution imaging of individual bacteria, and the nano/picoliter volume of microfluidic chip provides a stress-free culture environment during long incubation times. For the ease of analysis, microorganism isolation based on isolating 0 and 1 cells in each chamber (FIG. 7; 11). To do so bacteria concentration, flow rate and syringe infusion period are optimized.

It is also found that when bacteria seeding concentration increased, multiple cells were randomly isolated in channel. Although the analysis of multiple cells is difficult, it allows investigating the effect of initial bacterium number on antibiotic susceptibility (FIG. 12) or testing different microbiology applications related to quorum sensing.

The present invention is a versatile tool that is suitable for analysis of fluorescence and phase-contrast micrographs. For individual cell segmentation of micrographs and movies taken from fluorescence microscopy, intensity threshold, object circularity and object size are set as 400-4000 a.u., 0.67-1.00, and 2-6 μm. For individual cell segmentation of micrographs and movies taken from phase-contrast microscopy, intensity threshold, object circularity and object size are set as 1650-2260 a.u., 0.67-1.00, and 2-6 μm. The present microfluidic chip facilitates the determination of elongation, filamentation and motility of an individual rod-shaped bacterium, linked to the septation or stress response.

The multi-parametric motion-analysis incorporating line length, elongation ratio, circularity, line speed, path speed and intensity are found useful for the identification of resistance and susceptibility of E. coli in 4 hours. FIG. 15 showed that 0.25 μg/ml GM inhibits bulky E. coli GFP growth on agar plates while 0.1 μg/ml GM inhibits growth of ˜90% E. coli GFP. However, resistant subpopulation continues growing after 4 h.

### Example 4: Operation of the Miniature Clone Picking Device

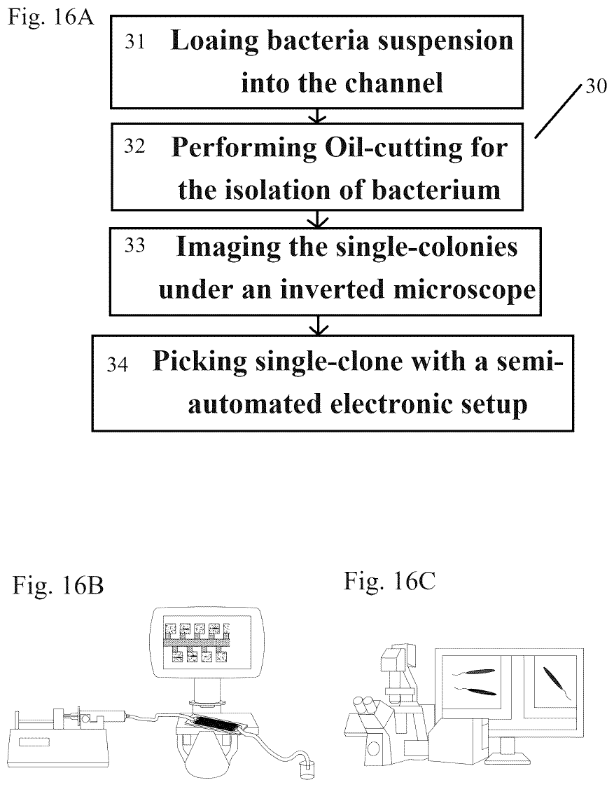

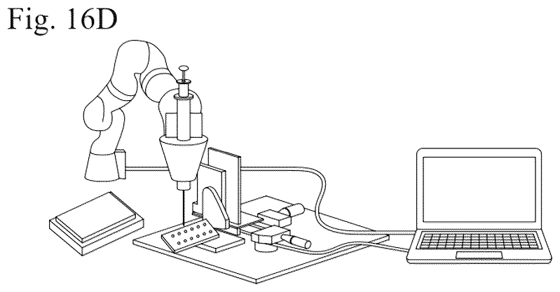

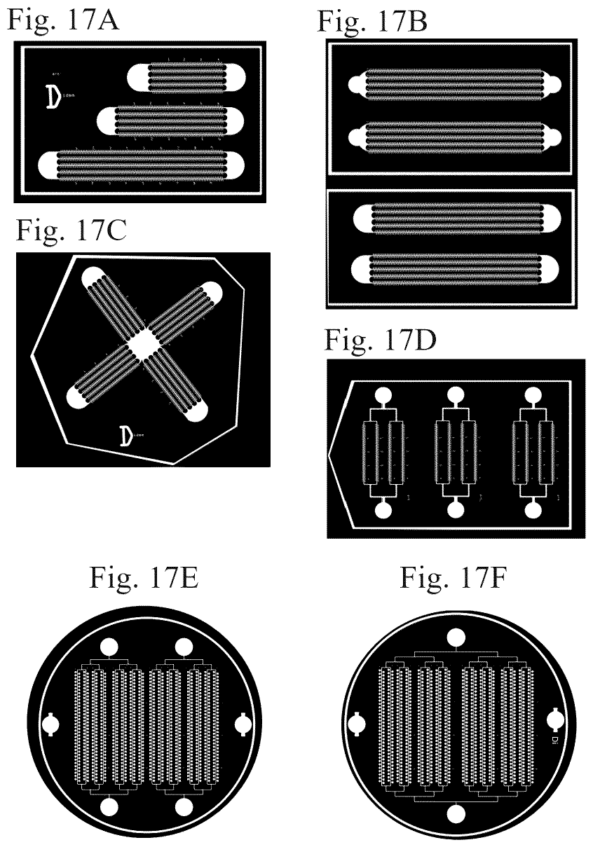

The present invention introduces a clone picking device and method (30) designed to operate within a small biosafety cabinet. The device offers advantages in reducing background signal and accurately tracking bacterial trajectories, crucial for various tasks including isolation, identification, and antibiotic testing. Placing the miniature clone picking device on the workstation within a small biosafety cabinet in the laboratory. Connect all necessary peripherals, including the inverted microscope and the semi-automated electronic picking system. Ensure proper connection and calibration of all devices (FIG. 16). The operation method described below.

Setup: Place the miniature clone picking device on the workstation within a small biosafety cabinet, ensuring proper connection and calibration of all peripherals, including the inverted microscope and semi-automated electronic picking system.

Loading Bacteria Suspension (31): Using a micropipette, load the suspension containing the target bacteria into the device's channel. Ensure the suspension flows uniformly through the channel, adjusting the flow rate as necessary for optimal performance.

Oil-Cutting Process (32): Following the loading of the bacteria suspension, introduce a layer of oil over the suspension within the channel. Initiate the oil-cutting process to create a barrier for isolating individual bacteria or colonies. The chip design aids in reducing background signal and precisely tracking bacteria trajectories during this phase.

Imaging Single Colonies (33): Once the oil-cutting process is complete, utilize the integrated inverted microscope to capture clear and detailed images of the isolated single colonies. Adjust microscope settings to optimize imaging quality. The device's design, particularly the microfluidic chip, ensures a stable imaging environment with minimal background noise.

Picking up single clone (34): a semi-automated electronic setup involves utilizing a specialized system to select and isolate individual colonies or clones from a bacterial suspension. The process typically begins by loading the bacterial suspension into a microfluidic channel or similar setup. Once loaded, the system employs electronic controls to guide a picking mechanism, such as a micro-needle or robotic arm, to target and isolate a single colony.

The electronic setup operates based on predefined parameters, which may include criteria such as colony size, shape, or optical properties. These parameters are often adjustable to accommodate different experimental needs. As the system picks a colony, sensors or imaging technology may provide feedback to ensure precise targeting and isolation.

### Example 5: Potential Microfluidic Single-Isolation-Imaging (SII-Chip) Chip Designs

The length of the microchannels varies in the range of 5-15 mm, to be constructed on a ϕ=20 mm coverslip bottom of a cell culture dish (similar to the framed area on the (black/white) mask designs).

The width of the channels varies in the range of 15-150 μm. The width (W) and the length (L) of the pico-isolation chamber varies in the range of W: 20-50 μm and L: 50-500 μm, or in a circular chamber design diameter varies in the range of 100-300 μm, which is suitable for clone picking with a 160-300 μm needle tip. The width of the chamber connection can be adjusted between 25-50 μm, while the width of the main channel can vary from 25 μm to 150 μm. The height of the single-isolation imaging chamber is alterable within the range of 1-14 μm depending on the bacteria of interest, and the height of the main channels (oil-liquid cutting) varies in the range of 5-20 μm.

The total number of single-isolation imaging chamber varies in the range of 50-5000, depending on the length, and the number of branch channels in the framed area, according to the dimensions of the substrate 0.10-0.17 mm thickness of coverslip glass bottom.

Microfluidic chip that allows individual-bacterium tracking offers several advantages for drug susceptibility testing: (i) it can provide continuous and real-time monitoring of bacterial responses to antibiotics, allowing for dynamic measurements of drug efficacy and resistance over time. (ii) the ability to manipulate and analyze a single bacterium enables precise measurements of antibiotic resistance, persistence and monoclonal heteroresistance compared to traditional bulk assays that solely rely on bulky growth. (iii) it can automate and parallelize the testing process, enabling the simultaneous screening of multiple bacteria in isolated chambers. (iv) the small sample volumes and automated testing can reduce the cost of reagents and labor compared to traditional methods. (v) it can provide controllable microenvironment and conditions for each individual bacterium, leading to more reproducible results. (vi) it allows dynamic phenotypic antibiotic drug susceptibility testing using single-cell microfluidics can provide more accurate and efficient assessment of antibiotic efficacy, helping to guide personalized antibiotic treatments and combat antibiotic resistance.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A fluidic chip for microorganism detection and analysis comprising:
a top layer,
wherein the top layer comprises:
at least one main channel facilitates the flow of a sample solution,
a plurality of branch channels is configured for facilitates the flow of oil,
a plurality of observation chambers is utilized for observing a single sample in the sample solution,
a plurality of connection channels connects the branch channel and the observation chambers.

2. The fluidic chip of claim 1, wherein the main channel further comprising:
a) an inlet through which the sample solution is introduced to the fluidic chip.
b) an outlet through which the sample solution is removed from the fluidic chip.

3. The fluidic chip of claim 1, wherein the main channel splits into 2-8 branch channels.

4. The fluidic chip of claim 1, wherein the main channel spans width ranging from 1-2 mm.

5. The fluidic chip of claim 1, wherein the branch channel spans width ranging from 5-150 μm and height ranging from 5-20 μm.

6. The fluidic chip of claim 1, wherein the connection channel is configured to permit the passage of the single sample in the sample solution.

7. The fluidic chip of claim 1, wherein the connection channel spans width ranging from 0.25-50 μm.

8. The fluidic chip of claim 1, wherein the observation chamber is configured for trapping the single sample.

9. The fluidic chip of claim 1, wherein the observation chamber can be one of the following shapes: rectangular prism-shaped, pentagonal prism-shaped, heptagonal prism-shaped, octagonal prism-shaped, triangular prism-shaped, square prism-shaped, cylindrical prism-shaped and trapezoidal prism-shaped.

10. The fluidic chip of claim 1, wherein the observation chambers spans height ranging from 1-14 μm.

11. The fluidic chip of claim 1, wherein the observation chamber spans 50-100 μm in width and 50-200 μm in length for a rectangular prism shape chamber.

12. The fluidic chip of claim 1, wherein the observation chamber spans ranging from 100-300 μm diameter for a cylindrical shaped chamber.

13. The fluidic chip of claim 1, wherein the fluidic chip spans 6-20 mm in length.

14. The fluidic chip of claim 1, wherein the top layer is composed of the following materials: polydimethylsiloxane (PDMS), polycarbonate (PC), polymethyl methacrylate (PMMA), polyolefin copolymer (POC), polystyrene (PS), polypropylene (PP) plastics, and hydrogel.

15. The fluidic chip of claim 1, wherein the top layer is disposed on a glass bottom layer.

16. The fluidic chip of claim 1, wherein the sample solution is composed of any one of the following species or combinations thereof: bacteria, fungi, archaea, protists, eukaryotes.

17. A method for utilizing the fluidic chip according to claim 1 for single microorganism isolation comprising:
a) Filling fluidic chip with a growth medium and the sample solutions at 0.0001-0.1 ml/h and 0.0001-0.3 ml/h flow rate, respectively;
b) Conducting oil-liquid cutting by oil infusion by a syringe pump at 0.001-0.1 ml/h flow rate within 15-60 sec to trap 0 or 1 microorganism inside the observation chamber.

18. The method of claim 17, further comprising
c) monitoring the single sample in the sample solution at temperature-controlled chamber with 37° C.

19. The method of claim 17, further comprising
d) prior to operation, the top layer is immersed in 1-10XPBS overnight and 500 μl 1-10XPBS is added to the bottom layer.

20. The method of claim 17, wherein the sample solution comprises bacteria with a concentration ranging from 5×10³-5×10⁴ CFU/ml.
